(19) United States
(12) Patent Application Publication
(10) Pub. No.: US 2016/0327539 A1
(43) Pub. Date: Nov. 10, 2016

(54) SIMPLIFIED APPARATUS AND METHOD FOR SOIL LYSIMETRY

(71) Applicant: GB Enterprises LLC, Santa Maria, CA (US)

(72) Inventors: Eryn Gray, Santa Maria, CA (US); Ned Brandt, Santa Maria, CA (US)

(21) Appl. No.: 14/704,163

(22) Filed: May 5, 2015

(51) Int. Cl. G01N 33/24; G01N 1/14

(52) U.S. Cl. CPC G01N 33/24; G01N 1/14

(57) ABSTRACT

A simplified lysimeter has a cylindrical body with a ceramic cup tip, which is porous for ingress of soil solution into a cavity in the body, attached at a bottom end of the body. A cap closes an upper end of the body and a single dip tube protrudes through and is sealed at the cap. A brace valve is attached to the dip tube and a hose is attached to the brace valve. The hose is removably in flow communication with a pump or a sample vessel.

### BACKGROUND

1. Field

This invention relates generally to the field of apparatus and methods for determining crop nutrients and more particularly to simplified lysimeter employing a single vacuum/pressure tube with a method of evacuation for ingress of soil solution into the lysimeter and pressurization for expulsion of the accumulated soil solution from the lysimeter for analysis.

2. Description of the Related Art

Lysimeters are used to measure and monitor soil contaminants and to monitor moisture levels and crop nutrient in the soil solution. 85-95% of all nutrients a crop takes in, come from the soil solution. The soil solution is made up of three contributing factors; the soil release of its chemical and biological releases of nutrient and salts, the nutrients and salts coming in from the irrigation water applied to the crop, and the fertilizer that growers apply. Collecting the soil solution with lysimeters allow analysis of the solution for any inorganic compounds including nutrients that the crops use for nutrition.

It is therefore desirable to provide a system and method for simplified soil solution measurement.

### SUMMARY

The embodiments disclosed herein provide a simplified lysimeter having a cylindrical body with a ceramic cup tip, which is porous for ingress of soil solution into a cavity in the body, attached at a bottom end of the body. A cap closes an upper end of the body and a single dip tube protrudes through and is sealed at the cap. A brace valve is attached to the dip tube and a hose is attached to the brace valve. The hose is removably in flow communication with a pump or a sample vessel.

The simplified lysimeter embodiment provides a method for obtaining soil solution for measurement by inserting the lysimeter at the predetermined depth. The hose is then connected to a vacuum/pressure pump and the pump is activated in a vacuum mode. A negative pressure is applied through the hose by opening the brace valve. When a sample is to be taken, the negative pressure is released on the lysimeter by opening the brace valve. Once the vacuum is released from the lysimeter, the hose is attached to the pump and a positive pressure is applied with the pump in a pressure mode through the hose of the lysimeter with the brace valve open. The brace valve is then closed while the pump is applying a positive pressure. The hose is disconnected from the pump and an outer end of the hose is placed a sampling vessel. The brace valve is opened whereby the positive pressure in the body of the lysimeter forces the soil solution out of the lysimeter through the dip tube that runs up the lysimeter and then through the brace valve and out through the hose into the sampling vessel.

### BRIEF DESCRIPTION OF THE DRAWINGS

These and other features and advantages of the present invention will be better understood by reference to the following detailed description of an exemplary embodiments when considered in connection with the accompanying drawings wherein:

FIG. 1 is a schematic representation of a simplified lysimeter embodiment as employed for the methods herein with associated components; and,

FIGS. 2A and 2B are a flow chart showing a representative method for lysimetry employing the disclosed embodiment of the simplified lysimeter.

### DETAILED DESCRIPTION

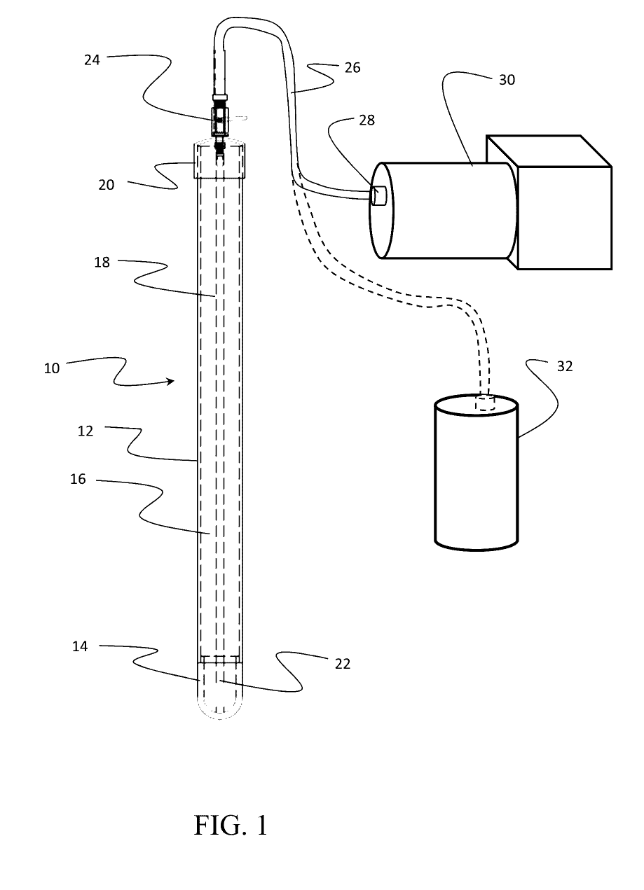

Embodiments shown in the drawings and described herein provide a lysimeter 10 as shown in FIG. 1. The lysimeter 10 incorporates a cylindrical body 12 with a ceramic cup tip 14 attached at a bottom end. The ceramic cup tip 14 is porous for ingress of soil solution into a cavity 16 in the lysimeter, as will be described in greater detail subsequently. A single dip tube 18 protrudes through and is sealed at a cap 20 closing an upper end of the body 12. The dip tube 18 extends to a termination 22 spaced from the ceramic cup tip 14 to provide an ullage. For exemplary embodiments, a 1″ gap is provided which is designed to minimize the loss of vacuum when soil conditions are dry by leaving a small amount of water in the cup tip as a seal. For exemplary embodiments, the body has an inner diameter of 2″ with a length including the ceramic cup tip 14 and cap 20 of between 8″ and 40″. A brace valve 24 is engaged to the dip tube 18 external to the lysimeter 10 and a hose 26 is connected to the brace valve 24 opposite the dip tube. The hose 26 terminates in a connector 28 which is removably attachable to a reversible vacuum/pressure pump 30 for fluid communication with the pump. While shown as a single dual mode (vacuum and pressure) pump, two separate pumps, one for vacuum and one for pressure may be employed. In exemplary embodiments, the hose is a ¼″ vacuum hose which may be a fiber or metal mesh reinforced flexible plastic hose. The connector 28 allows removal of the hose from the pump for insertion into or connection on a sampling vessel 32 for fluid communication with the sampling vessel.

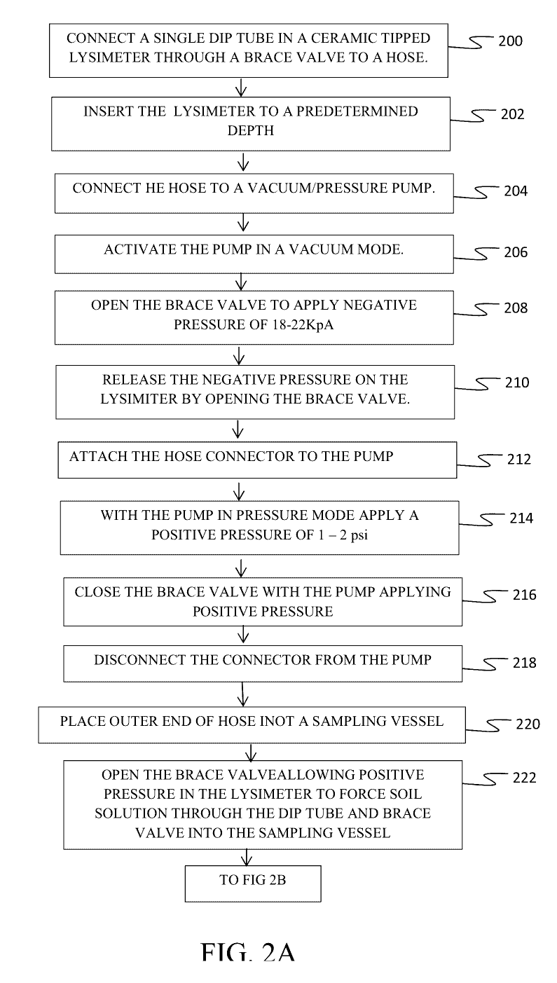

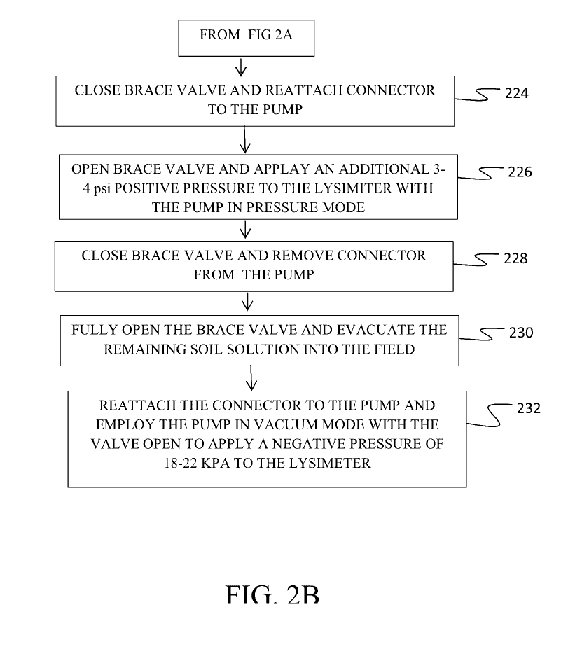

In operation, the lysimeter 10 is inserted into soil for which measurement is desired. The lysimeter may be inserted at a predetermined depth depending on desired measurement properties; 6″ to 12″ in to the soil to extract soil solution within the root zone (Rhizosphere) of the crop, 24″ into the soil to extract soil solution below the roots zone of the crop or 36″-48″ when developing leaching studies. The simplified construction of the lysimeter 10 with only a single dip tube 18 and single external connection of the hose 26 removably connected to either the pump 20 or the sampling vessel 32 allows an effective and simplified method of collection of soil solution as shown in FIG. 2.

A lysimeter is provided with a single dip tube connected through a brace valve to a hose, step 200. The lysimeter 10 is inserted at the predetermined depth, step 202. The hose 26 is connected to the vacuum/pressure pump 30, step 204, the pump is activated in a vacuum mode, step 206 and a negative pressure of 18-22 KpA (Ceuta bars) is applied through the hose by opening the brace valve, step 208. Multiple lysimeters may be employed as stations in an array distributed through the field being measured. Each station is then set to a sampling schedule which can range from every week, 2 weeks, 3 weeks to four weeks. When the lysimeter station is ready to be sampled the negative pressure on the lysimeter is released by opening the brace valve, step 210. Once the vacuum is released from the lysimeter the hose connector is attached to the pump, step 212, and, with the pump in a pressure mode, a positive pressure of 1-2 psi is applied through the hose of the lysimeter with the brace valve open, step 214. The brace valve is then closed while the pump is applying a positive pressure, step 216. The connector is disconnected from the pump, step 218 and the outer end of the hose is placed into the sampling vessel, step 220. Alternatively, the sampling vessel may be vented and have a mating connector for attachment of the hose. The brace valve is then opened, step 222, and the positive pressure in the body of the lysimeter forces the soil solution out of the lysimeter through the dip tube that runs up the lysimeter and then through the brace valve and out through the hose into the sampling vessel.

After the sample is taken, the brace valve is closed and the connector is reattached to the pump, step 224, and the brace valve is reopened and an additional 3-4 psi of positive pressure is applied to the lysimeter with the pump in pressure mode, step 226. The valve is closed and the pump removed, step 228. Then the valve is opened fully to evacuate the remainder of the soil solution that is in the lysimeter and the solution is pushed out of the lysimeter into the field, step 230.

After the lysimeter is evacuated from any excess fluid, the connector is reattached and the pump is employed in vacuum mode with the valve open to apply a negative pressure of 18-22 KpA (Ceuta Bars) to the lysimeter, step 232. The process starts over and then the station is sampled at the scheduled interval.

Coupled with plant nutrient uptake curves, fertility recommendations may be developed based off of the targeted nutrient the plants take in over a predetermined time frame, coupled with the analyzed soil solution that determines how much nutrition is already present in the soil solution and readily available to the crop. A highly effective and highly efficient fertilizing program may then be developed. This helps with minimizing ground water contamination and increases the nutrient uptake efficiency of the plants, resulting in an increase in yields and reduction in input cost the grower is investing in to produce a crop.

Having now described various embodiments of the invention in detail as required by the patent statutes, those skilled in the art will recognize modifications and substitutions to the specific embodiments disclosed herein. Such modifications are within the scope and intent of the present invention as defined in the following claims.

What is claimed is:

1. A simplified lysimeter system comprising:
a cylindrical body;
a ceramic cup tip attached at a bottom end of the body which is porous for ingress of soil solution into a cavity in the body;
a cap closing an upper end of the body,
a single dip tube protruding through and sealed at the cap;
a brace valve attached to the dip tube; and,
a hose attached to the brace valve and removably in flow communication with a pump or sample vessel.

2. The simplified lysimeter system as defined in claim 1 further comprising a connector attached to an end of the hose, said connector removably attachable to the pump.

3. The simplified lysimeter system as defined in claim 2 wherein the connector is removabley attachable to the sample vessel.

4. The simplified lysimeter system as defined in claim 1 wherein the dip tube terminates providing an ullage in the ceramic cup tip.

5. A method for obtaining soil solution with a simplified lysimeter for measurement comprising:
providing a lysimeter with a single dip tube connected through a brace valve to a hose;
inserting the lysimeter at the predetermined depth;
connecting the hose to a vacuum/pressure pump;
activating the pump in a vacuum mode;
applying a negative pressure through the hose by opening the brace valve;
releasing the negative pressure on the lysimeter by opening the brace valve;
once the vacuum is released from the lysimeter, attaching the hose to the pump;
applying a positive pressure with the pump in a pressure mode through the hose of the lysimeter with the brace valve open;
closing the brace valve while the pump is applying a positive pressure;
disconnecting the hose from the pump;
placing an outer end of the hose a sampling vessel; and, opening the brace valve whereby the positive pressure in the body of the lysimeter forces the soil solution out of the lysimeter through the dip tube that runs up the lysimeter and then through the brace valve and out through the hose into the sampling vessel.

6. The method of claim 5 further comprising:
after the sample is taken, closing the brace valve;
reattaching the hose to the pump;
reopening the brace valve and applying an additional positive pressure the lysimeter with the pump in pressure mode;
opening the valve fully to evacuate the remainder of the soil solution that is in the lysimeter;
and pushing the soil solution out of the lysimeter into the field.

7. The method of claim 6 further comprising:
after the lysimeter is evacuated from any excess fluid, employing the pump in vacuum mode with the valve open to apply a negative pressure to the lysimeter.
